(19) United States
(12) Patent Application Publication
(10) Pub. No.: US 2010/0075361 A1
(43) Pub. Date: Mar. 25, 2010

(54) METHODS OF GENERATING FLORESCENCE RESONANCE ENERGY TRANSFER (FRET) BETWEEN SEMICONDUCTOR QUANTUM DOTS AND FLUORESCENT DYES/PROTEINS VIA MULTI-PHOTON EXCITATION, ACHIEVING ZERO BACKGROUND OR DIRECT EXCITATION CONTRIBUTIONS TO THE FRET SIGNATURE

(75) Inventors: Hedi M. Mattoussi, Alexandria, VA (US); Igor L. Medintz, Springfield, VA (US); Aaron R. Clapp, Ames, IA (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(73) Assignee: Government of the United States of America the Secretary of the Navy(ONR/NRL), Arlington, VA (US)

(21) Appl. No.: 12/421,034

(22) Filed: Apr. 9, 2009

Related U.S. Application Data
(60) Provisional application No. 61/043,476, filed on Apr. 9, 2008.

(51) Int. Cl. C12Q 1/02; G01N 21/62; G06F 19/00

(52) U.S. Cl. 435/29; 436/171; 702/19; 977/774

(57) ABSTRACT

A system and method of sensing physiological conditions in biological applications includes a laser source for optically exciting a plurality of luminescent quantum dots and a plurality of biomolecules in a nanoscale sensing system having a nanocrystal structure, where the plurality of biomolecules is stained with dye. In a multi-photon excitation process, a laser system optically excites, the plurality of luminescent quantum dots and the plurality of biomolecules in the nanoscale sensing system, where fluorescence resonance energy transfer (FRET) occurs between the plurality of quantum dots and the plurality of biomolecules. Stability of self assembly of quantum dot peptide conjugates within the plurality of biomolecules is investigated. Physiological conditions at the cellular level are determined, using a spectrometer to sense fluorosence spectra. The sensing of physiological conditions includes transducing signals into immunoassays, clinical diagnostics and cellular imaging to provide treatment to biological subjects including human patients.

### RELATED APPLICATIONS

The present application herein is related to and claims the benefit of priority under 35 USC §119(e) of prior filed provisional patent application 61/043,476, which is herein incorporated by reference in its entirety.

### FIELD OF THE INVENTION

The present disclosure is generally related to the detection and/or sensing of chemical and biological agents potentially applicable to physiological media and tissue samples, fixed or living, by using a variety of optical excitations in the visible to near infrared light wavelengths. The sensing mechanism combined with the excitation method has potential uses in immunoassays, clinical diagnostics, and cellular imaging. In particular, the present disclosure describes, preferentially, optically exciting quantum dot donor fluorophores in systems where direct excitation of the acceptor fluorophore is undesirable, and where absorption of the excitation source by surrounding tissue must be minimized. By using two photon excitation, the limits of photophysical properties of organic dyes and fluorescent proteins are overcome, i.e., the florescence resonance energy transfer signal has an increased signal to noise ratio making the florescence resonance energy transfer signal easier to detect.

### BACKGROUND OF THE INVENTION

Presently, semiconductor quantum dots are ideal fluorophores for multi-photon excitation. The convergence of multi-photon excitation technologies using fluorophores with a florescence resonance energy transfer (hereafter “FRET”) process offers a versatile and effective strategy for developing sensitive assays and imaging applications. This builds upon previous work with quantum dot bioconjugates, where a florescence resonance energy transfer is routinely used as a means for determining molecular-scale changes (e.g., binding events, conformational rearrangement, etc.) near the nanocrystal surface.

Watt Webb and co-workers at Cornell University pioneered the used of multi-photon fluorescence microscopy since 1990. More recently, Webb's group reported two-photon excitation of commercially available water-soluble quantum dots. Webb's work showed that quantum dots have unusually large two-photon action cross-sections (˜10⁴ Goeppert-Mayer units) that are typically 2-3 orders of magnitude larger than organic fluorophores, which makes them ideal for deep-tissue imaging and related applications.

Luminescent quantum dots, with their large absorption cross sections, their superior photo and chemical stability, their broad excitation spectra, and their narrow emission bandwidths, are excellent alternatives to traditional organic dyes for fluorescence labeling and emerging nanosensing applications.

Using various surface functionalization techniques including cap exchange and encapsulation methods, quantum dots can be dispersed in aqueous media. This has naturally led to their use in biological applications, most notably in cellular labeling, and in the development of sensitive assays that can detect small molecules and oligonucleotides in solution.

It has been shown that that quantum dots are unique donor fluorophores for FRET where multiple acceptor dyes can be positioned around the quantum dot (hereafter “QD”) to substantially enhance the overall rate of FRET between QD and proximal dyes. Because of its exquisite sensitivity to changes in donor-acceptor separation distance (with sixth power dependence), FRET has proven to be a powerful method for detecting molecular scale interactions, such as binding events and changes in protein conformations. FRET-based QD-biomolecule sensing assemblies that are specific for the detection of target molecules including soluble TNT, DNA, and the activity of various proteolytic enzymes have been demonstrated.

Multi-photon fluorescence microscopy is the preferred high-resolution imaging method for thick (˜1 mm) tissue samples because of its intrinsic optical sectioning ability and limited out-of-focus photodamage. It also uses far red and near infrared excitation (650-1100 nm), which is ideally located in the tissue optical transparency window. However, FRET performance driven by two-photon excitation has been limited by the photophysical properties of organic dyes and fluorescent proteins. In particular, it is often difficult to devise a donor-acceptor pair with substantial spectral overlap for high FRET efficiency and non-overlapping two-photon absorption spectra for limited acceptor direct excitation. It has been shown that water-soluble semiconductor (and/or CdSe—ZnS) QDs are superior probes for multi-photon fluorescence imaging where typical QD two-photon action cross-sections are about one to two orders of magnitude larger than those of organic molecules designed specifically for such applications.

Multi-photon excitation of donor fluorophores in a florescence resonance energy transfer system has numerous advantages over a single photon excitation scheme; these advantages include the well-known benefits of superior optical sectioning due to a minimized excitation volume, relatively deep penetration into tissue samples, and limited photo-induced damage of surrounding tissue. In addition, multi-photon excitation of a donor fluorophore with a correspondingly large multi-photon absorption cross-section allows preferential donor excitation with minimal acceptor excitation. In a two-photon mode in contrast to the one-photon case, there is a substantial contribution from direct excitation of the acceptor in the one photon excitation mode but that contribution is negligible in the two-photon excitation mode. This is especially evident with quantum dot donors which have the highest known two-photon absorption cross-sections of any available fluorescent material. Single molecule imaging would also benefit from such an arrangement due to the reduced excitation volume allowing highly localized excitation of bioconjugates in vivo. Florescence resonance energy transfer efficiencies using one or two-photon excitation are effectively indistinguishable (see FIG. 3), which suggests that the underlying energy transfer mechanisms are unaffected by the specific excitation mode. The mechanistic equivalence between excitation modes in the florescence resonance energy transfer process is useful in the design, implementation, and data interpretation; it allows a direct comparison between results generated using the two excitation modes (i.e., one photon and two photon excitation modes).

The need exists for a variety of optical sensor designs that substantially improve in vivo sensing due to the transparency of tissue to near infrared wavelength light, by using the proper choice of an excitation source depending on the specific application, which can take advantage of the tunable optical properties of QD donors.

The need exists for a number of non-invasive methods for sensing physiological conditions inside the body.

The need exists for devising sensitive assays and performing real-time imaging of intracellular processes in immunoassays, clinical diagnostics, and cellular imaging.

The need exists for using multi-photon excitation in non-radiative energy transfer systems, where the underlying energy transfer mechanisms are unaffected by the specific excitation mode.

The need exists for combining recent advances made in quantum dot based fluorescence resonance energy transfer with advantages offered by multi-photon excitation of quantum dot dye pairs, such as the advantage of reduced photo oxidation.

The need exists for developing biosensors driven by FRET performance using two photon excitation that overcome the limits of the photophysical properties of organic dyes and fluorescent proteins.

In particular, the need exists to overcome difficulties in devising a donor-acceptor pair with substantial spectral overlap for high FRET efficiency and non-overlapping two-photon absorption spectra for limited acceptor direct excitation, where the two-photon excitation mode essentially eliminates the undesired direct excitation PL contribution common to the one-photon excitation case. This is due to a vastly reduced two-photon absorption cross-section (˜10⁴ smaller) for the dye relative to QDs (cf. Table 1), indicating that the entire observed Cy3 signal from the QD-dye conjugates (collected by the CCD detector) is attributed to non-radiative energy transfer.

The need exists for biosensors, where a decrease in QD steady-state PL and its radiative lifetime were essentially negligible when QDs were mixed with free dye (control samples) due to negligible FRET interactions

The need exists for developing systems and methods of preferentially exciting quantum dot donor fluorophores in systems, where direct excitation of the acceptor fluorophore is undesirable, and where absorption of the excitation source by surrounding tissue must be minimized.

The need exists for methods and systems for attachment of biomolecules (e.g., proteins, peptides, antibodies, etc.) to the surface of water-soluble quantum dots, which creates a stable hybrid nanoparticle with unique optical properties and biological functionality.

The need exists for efficient florescence resonance energy transfer between quantum dot donors and organic dye acceptors to serve as a signal transduction mechanism in a sensing arrangement.

The need exists for using the ability of florescence resonance energy transfer to provide a measurable optical signal corresponding to binding events and molecular rearrangements.

### SUMMARY OF THE INVENTION

A system and method of sensing physiological conditions in biological applications, using a multi-photon excitation system includes automated measuring instrumentation and a laser source for directly optically exciting a plurality of donors and acceptors, after preparing the plurality of acceptors in a nanoscale sensing system, having a nanocrystal structure. In the multi-photon excitation process, a pulsed and/or continuous wave laser system source directly optically excites, the plurality of donors and the plurality of acceptors previously prepared in the nanoscale sensing system. Based on the direct optical excitation, an energy transfer occurs between the plurality of donors and the plurality of acceptors at the cellular level. The multi-photon excitation process can be a two-photon excitation process, which yields a photoluminescence (PL) contribution. The stability of self assembly of the plurality of donors with the plurality of acceptors is investigated; and using a photodiode (i.e., photo detector), changes in the nanoscale sensing system are detected, where detecting changes in the nanoscale sensing system provide either nanocrystal biological specificity for specific targets or compatibility with biological environments. Physiological conditions at the cellular level are determined and/or sensed by using a spectrometer to sense fluorosence spectra. The sensing of physiological conditions includes transducing signals without corrupting influences into immunoassays, clinical diagnostics, and cellular imaging to provide treatment to biological subjects including human patients.

### BRIEF DESCRIPTION OF THE DRAWINGS

FIG. 1 illustrates a two-photon excited FRET in a CdSe—Zn QD bioconjugate system.

FIG. 2A illustrates emission spectra from Cy3 using one-photon excitation.

FIG. 2B illustrates emission spectra from Cy3 using two-photon excitation.

FIG. 3A illustrates a titration sequence showing one-photon emission spectra from a QD-MBP-Cy3 FRET system.

FIG. 3B illustrates a titration sequence showing two-photon emission spectra from a QD-MBP-Cy3 FRET system.

FIG. 4A illustrates a schematic of a QD-protein-dye conjugate and FRET driven by a two-photon excitation process, where deconvoluted PL spectra of QDs and Cy3 as a function of the number of MBP-Cy3 per QD using two-photon excitation, 540 nm QDs.

FIG. 4B illustrates a schematic of a QD-protein-dye conjugate and FRET driven by a two-photon excitation process.

FIG. 4C illustrates a schematic of a QD-protein-dye conjugate and FRET driven by a two-photon excitation process, where deconvoluted PL spectra of QDs and Cy3 as a function of the number of MBP-Cy3 per QD using two-photon excitation, 510 nm QDs.

FIG. 4D illustrates a schematic of a QD-protein-dye conjugate and FRET driven by a two-photon excitation process, where deconvoluted PL spectra of QDs and Cy3 as a function of the number of MBP-Cy3 per QD using two-photon excitation, 540 nm QDs.

FIG. 5A illustrates two-photon fluorescence microscopy images of HEK 293T/17 cells following incubation for 1 hour with 510 nm QDs conjugated to:

FIG. 5B illustrates one-photon fluorescence microscopy images of the same HEK 293T/17 cells corresponding to the conditions (A-C), respectively, (λₑₓ=488 nm, scale bar=20 μm).

FIG. 6A illustrates composite spectra from a reagentless sensing format using 510 nm QDs with MBP-Cy3 labeled at Cys41. The Cy3 PL signal decreases monotonically as a function of maltose concentration.

FIG. 6B illustrates transformation of the PL data versus maltose concentration for four different arrangements using 510 nm and 540 nm QDs with MBP-Cy3 using one and two-photon excitation modes. The titration curves demonstrate the equivalence of the sensing arrangements regardless of the QDs used or excitation mode chosen.

FIG. 8A illustrates a nanoscale system of sensing physiological conditions in biological applications at a cellular level, using optical sensors, as described in the method illustrated in FIG. 7A and FIG. 7B.

### DETAILED DESCRIPTION OF THE INVENTION

Preferred exemplary embodiments of the present disclosure are now described with reference to the figures, in which like reference numerals are generally used to indicate identical or functionally similar elements. While specific details of the preferred exemplary embodiments are discussed, it should be understood that this is done for illustrative purposes only. A person skilled in the relevant art will recognize that other configurations and arrangements can be used without departing from the spirit and scope of the preferred exemplary embodiments. It will also be apparent to a person skilled in the relevant art that this invention can also be employed in other applications. Devices and components, such as the laser, the photodetector and the readout devices described in the exemplary embodiments can be off the shell commercially available devices or specially made devices. Further, the terms “a”, “an”, “first”, “second”, and “third” etc. used herein do not denote limitations of quantity, but rather denote the presence of one or more of the referenced items(s).

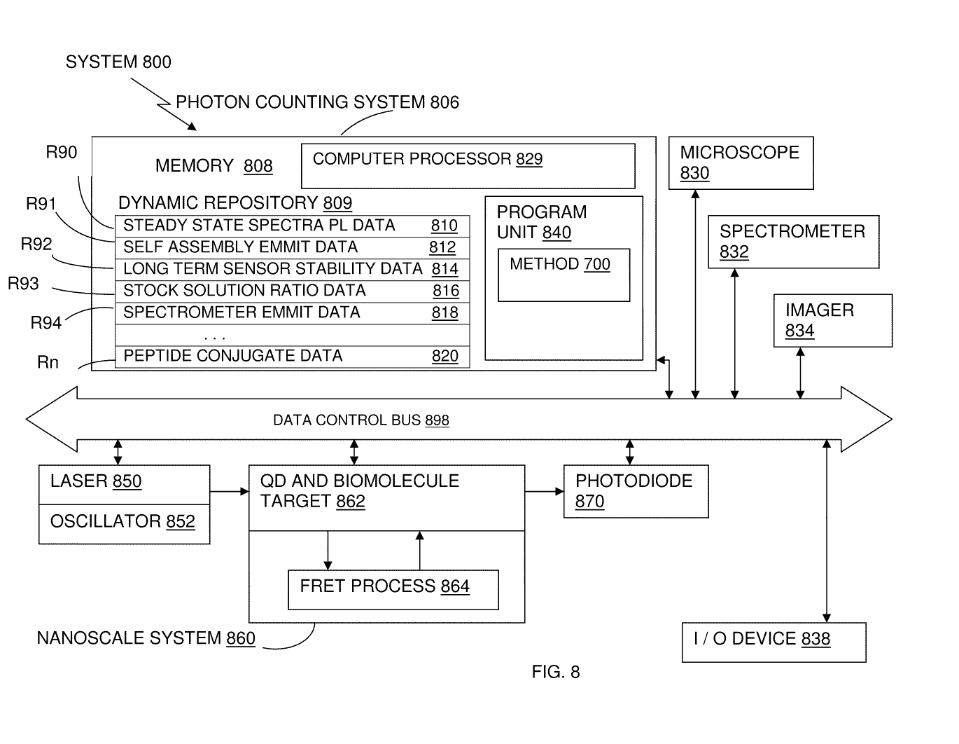

Exemplary embodiments describe a system and method of sensing physiological conditions in biological applications at a cellular level, using optical sensor technologies. Referring to FIG. 8, the system of sensing physiological conditions in biological applications at a cellular level, using optical sensor technologies 800 (hereafter “the system 800”) operates to implement methods of sensing physiological conditions in biological applications at a cellular level, using optical sensor technologies.

Referring to FIG. 7A, FIG. 7B and FIG. 8, in exemplary embodiments, a method of sensing physiological conditions in biological applications at the cellular level, using optical sensor technologies 700 (hereafter “the method 700”) is illustrated in FIG. 7A, FIG. 7B and further illustrated as implemented in FIG. 8, as method operations executed and/or conducted in association with the system 800 and implemented also in method operations executed and/or included in a program unit 840.

Referring again to FIG. 7A, FIG. 7B and FIG. 8, in exemplary embodiments, the system 800 includes a laser 850 and an oscillator 852. The laser 850 is communicatively coupled to a nanoscale system 860 (hereafter “the nanoscale system 860”) having a donor and/or an acceptor target 862. The nanoscale system 860 also implements an energy transfer process, such as a FRET process 864.

Referring again to FIG. 8, the system 800 includes a plurality of automated measuring instrumentation, including a photodiode 870, an imager 834, a spectrometer 832, a microscope 830 and a photon counting system 806. The plurality of automated measuring instrumentation may each include computer processors or may be connected to a stand along computer processor or may be controlled and/or accessed or in communication with and/or by any one or more of the above described computer processors, such as a computer processor 829, illustrated as part of the photon counting system 806. In exemplary embodiments, each instrument of the plurality of measuring instrumentation is communicatively coupled to the laser 850, the nanoscale system 860 the photodiode 870 and each other by a data control bus 898. In exemplary embodiments, human interface devices such as an input/output device 838 (hereafter “I/O device 838”) can be used to input data, parameters, values and formulas into the system 800 for use by the method 700 and the system 800.

Referring again to FIG. 8, in accordance with exemplary embodiments, the I/O device 838 can be at least one or more of a mouse, a keyboard, a touch screen terminal, a light pen wand, a joystick, a thumbwheel, a copier system or machine, a hardcopy paper scanner system or machine, a microphone or an electronic and/or a radio frequency scanning device or one or more biometric input and/or reading devices.

Referring again to FIG. 8, in accordance with exemplary embodiments, the computer processor counting system 806 further includes a memory 808 (hereafter “the memory 808”). Residing in the memory 808 are a program unit 840 (hereafter “the program unit 840”) and a dynamic repository 809 (hereafter “the dynamic repository 809”). Residing in the dynamic repository 809 are a plurality of repository entry locations R90, R91, R92, R93, R94, up to and including Rn, where Rn theoretically represents an infinite number of repository entry locations limited only by known physical and/or virtual memory capacity. Thus, each repository entry location R90 up to Rn can hold, store and/or save a plurality of information and/or data including data such as steady state spectra PL data 810, represented as stored in repository entry location R90; self assembly emit data 812, represented as being stored in repository entry location R91; long term sensor stability data 814, stored and/or saved in repository entry location R92; stock solution ratio data 816, held in repository entry location R93, spectrometer emit data 818, stored in repository entry location R94; and peptide conjugate data 820, saved in representative repository entry location Rn. These groups of data and information and other measurement data and parameters can be easily and programmatically accessed, exercised and for transducing fluorescence spectra signals representing physiological conditions into immunoassays, clinical diagnostics, and cellular imaging in order to provide treatment to biological subjects at the cellular level. In addition, a plurality of other data and information may be entered into the repository entry locations R90 through Rn, including mathematical rules and/or constraints. Furthermore, these groups of information and data, including the plurality of other data can be stored temporarily and/or permanently and/or semi permanently in the repository entry locations R90 through Rn. In exemplary embodiments, these groups of information and data can be downloaded programmatically over the data control bus 898 or entered manually by way of the I/O device 838.

In exemplary embodiments, at an operation start 702, the system 800 and method 700 of sensing physiological condition in biological applications using a multi-photon excitation system are activated electronically.

Referring to FIG. 7A, FIG. 7B and FIG. 8, in exemplary embodiments of the system 800 and the method 700, at an operation preparing a plurality of acceptors 704 (hereafter “the operation 704”), a plurality of acceptors in the nanoscale system 860 are prepared for direct optical excitation and energy transfer process.

In exemplary embodiments, the preparation of QD bioconjugates used is described herein using methods performed via a self-assembly between nanocrystals and protein/peptides appended with either a polyhistidine tract or a leucine zipper attachment domain in aqueous buffer. A small volume of water-soluble QD stock solution is added to buffer containing labeled biomolecules (e.g., protein, DNA).

In a first exemplary embodiment, the precise amounts of QD and biomolecule stock solutions added to the buffer are determined by the specific concentrations and the desired ratio of donor to acceptor. The self-assembly of histidine-terminated biomolecules onto dihydrolipoic acid (DHLA) capped QDs occurs rapidly (within a few minutes) at room temperature and produces stable bioconjugates. This is one possible procedure that can be used; however, the proposed FRET process is general to any bioconjugate system that results in an appropriate donor-acceptor pair in close proximity. Two-photon excitation of the sample is achieved using a pulsed laser source operating in the near infrared. A pulsed source is usually required to generate the high intensities necessary to realize a multi-photon excitation process.

In the first exemplary embodiment, in regard to quantum dot protein conjugates, engineered variants of E. coli maltose binding protein (MBP) were appended with a C-terminal polyhistidine tract (MBP-His) to allow metal-affinity driven self-assembly on the surface of DHLA-functionalized QDs. These proteins were also modified to contain single cysteine mutations (at positions D41C or D95C) for specific labeling with maleimide-functionalized Cy3 dye prior to conjugate assembly. MBP-His labeled at D95C was used for the steady state and time-resolved fluorescence experiments, while MBP-His labeled at D41C was exclusively used in the reagentless sensor design. The overall MBP-to-QD ratio was maintained at a 15:1 ratio, thus ensuring essentially a complete coverage of the QD and maintaining a consistent QD quantum yield.

In the first exemplary embodiment, in regard to quantum dot peptide conjugates, cell penetrating peptide (CPP) was synthesized by Boc-solid phase peptide synthesis with the sequence (His)₈-Trp-Gly-Leu-Ala-Aib-Ser-Gly-(Arg)₈-amide, where Aib is the artificial residue alpha-amino isobutyric acid. The Cy3-labeled peptide was synthesized with the sequence, Ac-(His)₆-Gly-Leu-Aib-Ala-Ala-Gly-Gly-His-Tyr-Gly-Cys-NH₂, where Ac is an acetyl group at the N-terminus. This peptide was labeled with a maleimide-functionalized Cy3 at the cysteine residue. The polyhistidine sequences at the N-termini of the peptides allowed and/or caused their self-assembly on the QD surface. QD conjugate stock solutions were prepared by incubating 1 μM of DHLA-capped QDs with the appropriate ratios of peptides, i.e., 60 CPP/QD for CPP-QD conjugates, or 60 CPP mixed with 2 peptide-Cy3 per QD for CPP-QD-peptide-Cy3 conjugates.

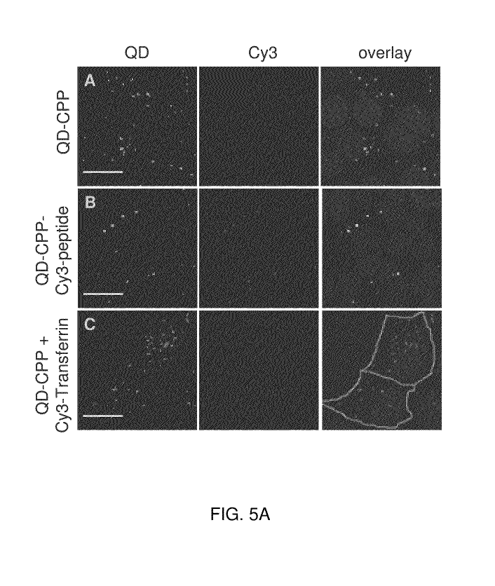

FIG. 5A (top panels) shows two-photon fluorescence microscopy images of HEK 293T/17 cells following incubation for 1 hour with 510 nm QDs conjugated to: (A,C) CPP, (B) CPP and 2 Cy3-labeled peptides; CPP-QD conjugates were prepared with 60 CPP per QD. In (C), the cells were also incubated with Cy3 labeled-transferrin (not bound to QDs). The QD staining is entirely located in the cell cytoplasm as demonstrated by the overlaid image in (C); representative cell membranes are outlined in white. (λₑₓ=840 nm, scale bar=20 μm). Rather high concentrations of DAPI nuclear staining agent (in comparison with QD and dye) were used to allow and/or cause easy visualization of the cell nucleus in two-photon excitation mode.

In FIG. 5A, insets (A,C) CPP, inset (B) CPP and 2 Cy3-labeled peptides; CPP-QD conjugates were prepared with 60 CPP per QD. In inset (C), the cells were also incubated with Cy3 labeled-transferrin (not bound to QDs). The QD staining is entirely located in the cell cytoplasm as demonstrated by the overlaid image in inset (C); representative cell membranes are outlined in white, (λₑₓ=840 nm, scale bar=20 μm). Rather high concentrations of DAPI nuclear stating agent (in comparison with QD and dye) were used to allow easy visualization of the cell nucleus in two-photon excitation mode system configuration of an auto analyzing stage of an electro magnetic field measuring system.

FIG. 5B (bottom panels) shows one-photon fluorescence microscopy images of the same HEK 293T/17 cells corresponding to the conditions (A-C), respectively. (λₑₓ=488 nm, scale bar=20 μm.).

Referring to FIG. 7A, FIG. 7B and FIG. 8, at an operation directly optically exciting a plurality of donors and acceptors 706 (hereafter “the operation 706”), a plurality of donors and the plurality of acceptors in the nanoscale sensing system 860 are directly, optically excited with the laser 850. The laser 850 includes either pulsed and/or continuous wave excitation sources.

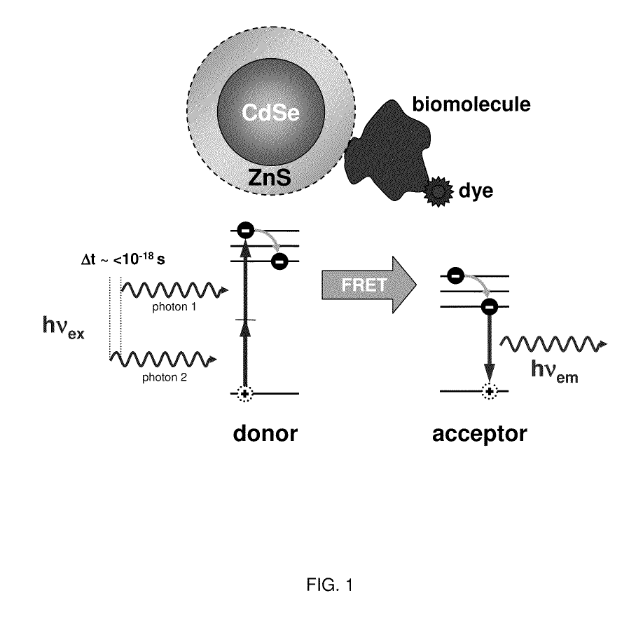

Other possible exemplary embodiments have common sources including Ti:Sapphire (tunable emission between 650 and 1100 nm) and Nd:YAG (1064 nm emission) lasers operating in pulsed mode, although it is conceivable that any pulsed laser source operating to the red of the QD absorption band edge could be used as a multi-photon excitation source. The proper choice of an excitation source depends on the specific application, but there are many possible options available especially considering the tunable optical properties of QD donors. A schematic depicting the bioconjugate system and the associated excitation/energy transfer mechanism is shown in FIG. 1.

In the first exemplary embodiment, concerning two-photon excited steady state spectra, two-photon excitation was generated using tunable Ti:sapphire laser (200 fs pulse width, 76 MHz, Clark-MXR, Dexter, Mich.) operating at 800 nm and focused with an objective lens to a spot within a quartz cuvette containing the bioconjugates sample. Fluorescence spectra were collected using a spectrometer.

In the first exemplary embodiment, in regard to fluorescence lifetimes, the excitation source for the time-resolved experiments used the pulse-picked output (5 MHz) of a mode-locked Ti:Sapphire oscillator (pulse width<200 fs, Mira 900, Coherent, Santa Clara, Calif.) with a center wavelength at 800 nm.

Referring to FIG. 7A, FIG. 7B and FIG. 8, at an operation transferring energy between the plurality of donors and the plurality of acceptors 708 (hereafter “the operation 708”), resulting from the operation 706 of directly exciting the plurality of donors and acceptors, an energy transfer occurs between the donors and acceptors. In exemplary embodiments, there are a plurality of different types of energy transfer processes suitably implemented in the operation 708. In exemplary embodiments, some relevant energy transfer processes include (1) Fluorescence (Förster) resonance energy transfer (i.e., FRET), which is characterized as dipole-dipole interactions through space; and (2) Dexter electron transfer, which is characterized as migration of an excited electron into a donor material; and (3) Surface energy transfer, which is characterized as dipole-metallic surface interactions through space, similar to FRET. In exemplary embodiments, Dexter and FRET are the two main implementations, and others are largely specific implementations of Dexter and FRET, but with additional similar properties, that is to say, that in exemplary embodiments surface energy transfer (SET and/or NSET occurs between a fluorescent material and a metal, however, the energy transfer mechanism is substantially similar to FRET.

In exemplary embodiments, in the FRET process 864, methods and systems in the method 700 implemented in the system 800 use a generalized self-assembly approach, in the attachment of biomolecules (e.g., proteins, peptides, antibodies, etc.) to the surface of water-soluble quantum dots, which creates a stable hybrid nanoparticle with unique optical properties and biological functionality. Efficient fluorescence (or Förster) resonance energy transfer (hereafter “FRET”) between quantum dot (hereafter “QD”) donors and organic dye acceptors have been repeatedly demonstrated to serve as a signal transduction mechanism in a sensing arrangement. Thus, the ability of FRET to provide a measurable optical signal corresponding to binding events and molecular rearrangements is accomplished.

QD based fluorescence resonance energy transfer is advantageous in multi-photon excitation of quantum dot dye pairs, because of reduced photo oxidation effects attributed to the QD donors. Thus, photo oxidation of donors bound with biomolecules is minimized to a degree of being negligible.

The FRET between QD donors and organic dye acceptors, is herein disclosed, as driven by a two-photon process using sub-band excitation energy (far red and near infrared photo-excitation). The FRET process between QDs and proximal dyes using this format has two unique features: 1) it exploits the very high two-photon action cross-sections of QDs compared to those of conventional dyes, which results in a near-zero background contribution from the dye acceptors due to direct excitation, independent of the excitation wavelength; and 2) it provides high signal-to-background ratios in FRET imaging of cells and tissue samples by substantially reducing both autofluorescence and direct excitation contributions to the acceptor photoluminescence (PL) signal. These features can considerably simplify data analysis, in particular when signals of both QD donor and dye acceptor are required to interpret assay results; they can also improve applications such as intracellular FRET sensing and imaging. In exemplary embodiments, the energy transfer resulting from two-photon excitation is entirely consistent with results collected using one-photon excitation (which uses higher energy photo-excitation), and in agreement with predictions of the Förster theory.

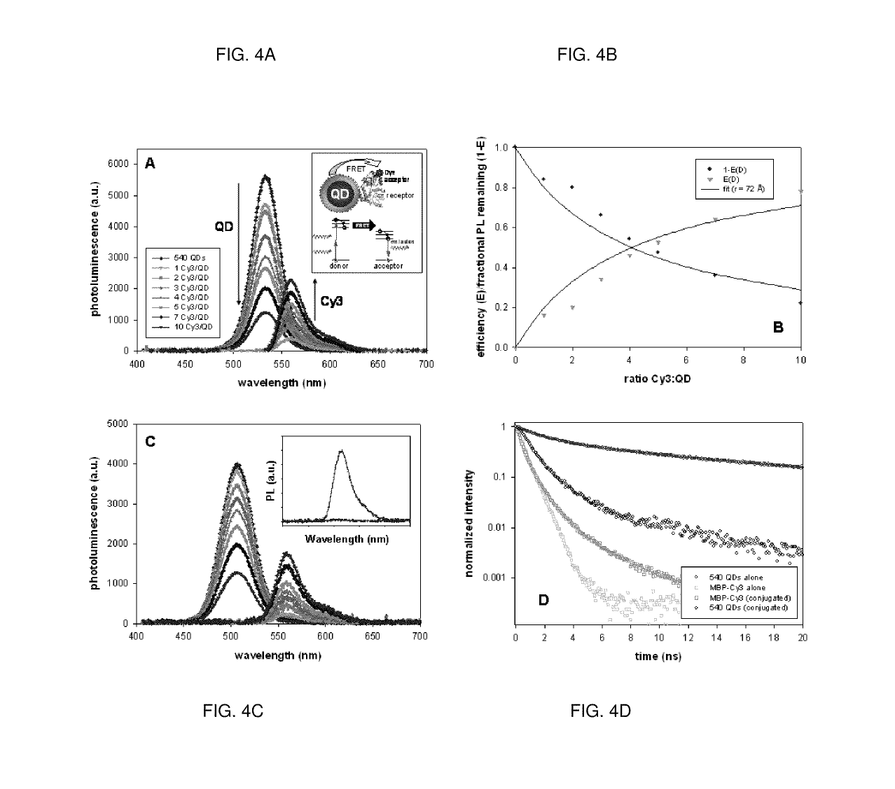

Table 1 shows the two-photon action cross-section values measured for our QDs in toluene and in water solutions, along with those of traditional dyes. Data show that the measured values for QDs are 2-3 orders of magnitude higher than Cy3 or those reported in the literature for other common organic dyes. Measured values are comparable to the two-photon action cross-sections reported. Steady-state composite spectra collected from samples under two-photon excitation were deconvoluted to yield individual photoluminence (PL) contributions using the known spectral profiles of isolated QDs and Cy3 dye emissions (see FIG. 4A, FIG. 4B, FIG. 4C and FIG. 4D).

FIG. 4A illustrates a schematic of a QD-protein-dye conjugate and FRET driven by a two-photon excitation process, where deconvoluted PL spectra of QDs and Cy3 as a function of the number of MBP-Cy3 per QD using two-photon excitation, 540 nm QDs. In particular, the inset in (A) shows a schematic of a QD-protein-dye conjugate and FRET driven by a two-photon excitation process.

FIG. 4B illustrates a schematic of a QD-protein-dye conjugate and FRET driven by a two-photon excitation process, where deconvoluted PL spectra of QDs and Cy3 as a function of the number of MBP-Cy3 per QD using two-photon excitation, with fractional PL (compared to unlabeled QD-conjugate solutions) and FRET efficiencies E for 540 nm QDs.

FIG. 4C illustrates a schematic of a QD-protein-dye conjugate and FRET driven by a two-photon excitation process, where deconvoluted PL spectra of QDs and Cy3 as a function of the number of MBP-Cy3 per QD using two-photon excitation, 510 nm QDs. In particular, the inset in (C) shows a comparison between FRET-induced Cy3 PL (purple) and contribution due to direct two-photon excitation collected for a control MBP-Cy3 sample (crimson).

FIG. 4D illustrates a schematic of a QD-protein-dye conjugate and FRET driven by a two-photon excitation process, where deconvoluted PL spectra of QDs and Cy3 as a function of the number of MBP-Cy3 per QD using two-photon excitation, 540 nm QDs. Furthermore, the inset in (D), shows time-resolved fluorescence decays for 540 nm QD-MBP-Cy3 where PL signals from donor and acceptor are spectrally isolated by appropriate band pass filters. Decay profiles are shown for isolated QDs, isolated MBP-Cy3, and for each fluorophore when brought together in a conjugate. Average lifetimes were estimated from fits of the experimental data to biexponential decays for all solutions. Data from the Cy3 solution were well fit to a single exponential decay function.

Referring to FIG. 7A, FIG. 7B and FIG. 8, at an operation investigating stability of self assembled donors and the plurality of acceptors 710 (hereafter “the operation 710”), the plurality of measuring and/or measurement instrumentation can be used to investigate, measure and/or detect the effects of transferring energy between donors and acceptors, based on directly exciting the donors and acceptors.

In exemplary embodiments, the PL contribution due to direct excitation was measured using control samples containing MBP-Cy3 only and subtracted from the composite spectra before analysis. The QD PL spectra collected using a two-photon excitation mode maintain the same symmetric and narrow features as those collected in the one-photon excitation mode. Time-resolved fluorescence measurements showed a pronounced decrease in QD donor lifetime for solutions of QD-MBP-Cy3 compared with unconjugated nanocrystals. The data also indicate that the Cy3 lifetime increases in the QD conjugate as compared to Cy3 alone. This observation is consistent with results using a one-photon excitation mode. A decrease in QD steady-state PL and its radiative lifetime were essentially negligible when QDs were mixed with free dye (control samples) due to negligible FRET interactions.

FIG. 4A and FIG. 4B (top panels) show two-photon fluorescence microscopy images of HEK 293T/17 cells following incubation for 1 hour with 510 nm QDs conjugated to: (A,C) cell-penetrating peptides (CPP), (B) CPP and 2 Cy3-labeled peptides; CPP-QD conjugates were prepared with 60 CPP per QD. In (C), the cells were also incubated with Cy3 labeled-transferrin (not bound to QDs). The QD staining is entirely located in the cell cytoplasm as demonstrated by the overlaid image in (C); representative cell membranes are outlined in white. (λₑₓ=840 nm, scale bar=20 μm). Rather high concentrations of diamidino-2-phenylindole (DAPI) nuclear staining agent (in comparison with QD and dye) were used to allow easy visualization of the cell nucleus in two-photon excitation mode. (bottom panels) One-photon fluorescence microscopy images of the same HEK 293T/17 cells corresponding to the conditions (A-C), respectively (λₑₓ=488 nm, scale bar=20 μm.).

TABLE 1

Two-photon action cross-
section (GM, 800 nm)

| | Water | toluene |
|---|---|---|
| 510 nm QDs | 8500 | — |
| 540 nm QDs | 13800 | 15000 |
| 555 nm QDs | 23200 | 19500 |
| 570 nm QDs | — | 23000 |
| Cy3 | <1 | — |
| Fluorescein | 36 | — |
| GFP | 6 | — |

Table 1 contains data describing two-photon action cross-sections for QDs populations in water and toluene, characterized using fluorescein as a standard in aqueous buffer (φσ₂ₚ=36 GM at 800 nm and pH 13), Cy3, fluorescein and wild-type green fluorescent protein (GFP) (from reference 28). Although multi-photon absorption profiles are non-linear optical phenomena, the longer wavelength emitting QD populations show higher two-photon action cross-sections, consistent with the one-photon absorption behavior.

The results above indicate that the loss of QD emission is specifically due to self-assembly of the dye-labeled protein on the nanocrystal surface, which positions the dye acceptors close to the QD donor. This culminates in efficient non-radiative transfer of excitation energy from the QD to the proximal Cy3 dye (see FIG. 4). The experimental FRET efficiency can be readily determined using the relation (developed for one-photon excitation FRET):

$$E = 1 - \frac{{F}_{DA}}{{F}_{D}} , \qquad ( 1 )$$

where F_{DA} and F_{D} are the QD PL measured in the presence and absence of dye acceptors, respectively.^{[26]} FIG. 4 shows that the FRET efficiency increases with the number of dyes positioned near the QD surface, n. The data also show that the dependence of the FRET efficiency on the ratio n for the two-photon excitation mode is indistinguishable from the results using one-photon excitation. The enhancement in the FRET efficiency with an increasing number of dyes is due to the increased effective overlap integral when multiple acceptors interact with a central QD donor. However, the two-photon excitation mode essentially eliminated the undesired direct excitation PL contribution common to the one-photon excitation case. This is due to a vastly reduced two-photon absorption cross-section (˜10⁴ smaller) for the dye relative to QDs (cf. Table 1), indicating that the entire observed Cy3 signal from the QD-dye conjugates (collected by the CCD detector) is attributed to non-radiative energy transfer. This is a desirable advantage over a one-photon mode where direct excitation of the acceptor, though generally small for an optimized excitation wavelength, must be carefully subtracted from the composite spectra to properly estimate the FRET efficiency. Analysis of the QD PL loss (or E) using Förster's formalism provided estimates of donor-acceptor distances r for both assemblies using the formula executed in an automated analysis and computer processing system:

$$r = {{R}_{0} [ \frac{n ( 1 - E )}{E} ]}^{1 / 6} , \qquad ( 2 )$$

where R₀ is the Förster distance. The 510 nm QD-MBP-Cy3 and 540 nm QD-MBP-Cy3 titration results yielded fitted distances r of 67±5 and 72±4 Å, respectively, consistent with the known sizes of the QD bioconjugates and with values extracted using one photon excitation mode. Equivalence of the FRET processes using one and two-photon excitation modes indicates that the transition dipole of the excited QD donor, which is responsible for non-radiative transfer of excitation energy, has the same properties regardless of the photo-excitation mechanism. Furthermore, the point dipole approximation is sufficient for describing QD fluorophores in this arrangement.

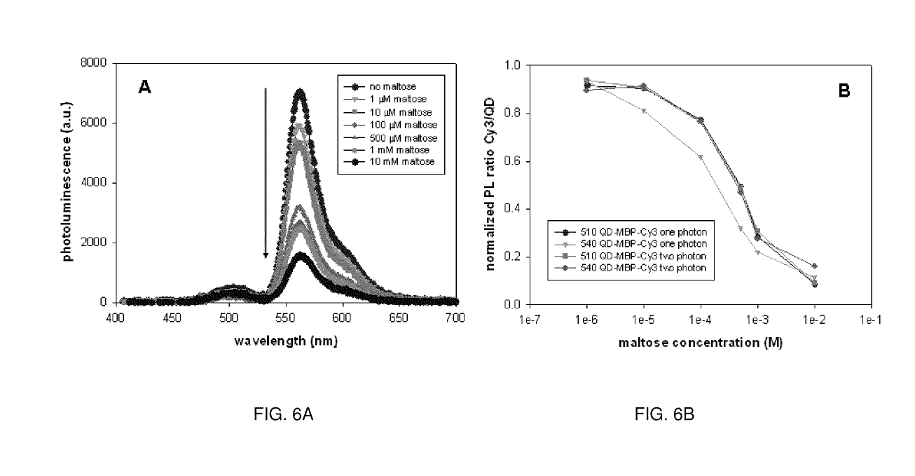

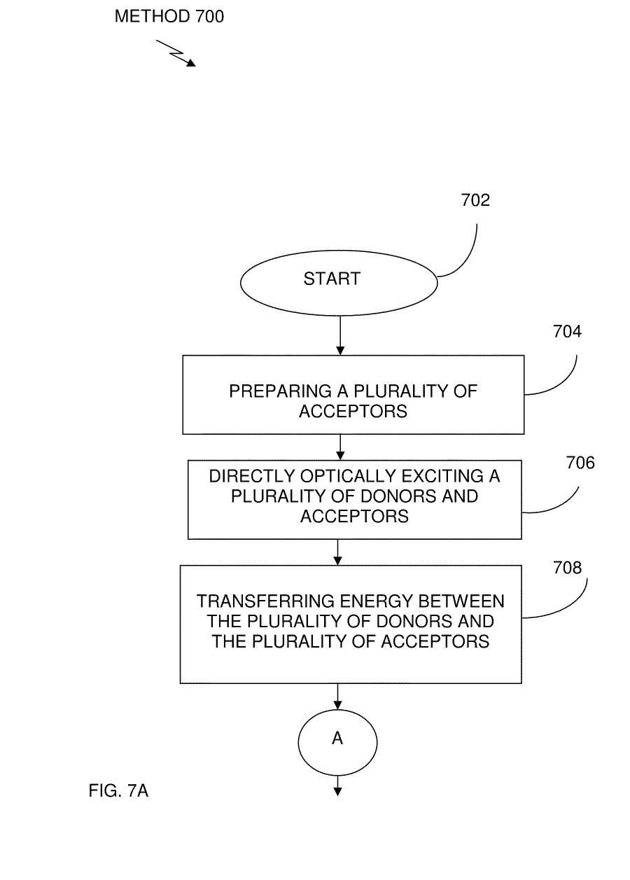

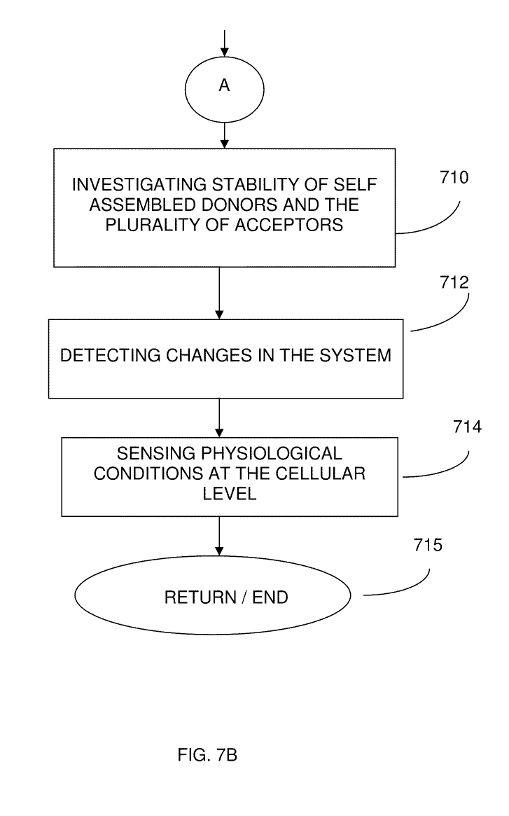

FIG. 6A shows composite spectra from a reagentless sensing format using 510 nm QDs with MBP-Cy3 labeled at Cys41. The Cy3 PL signal decreases monotonically as a function of maltose concentration.

FIG. 6B shows transformation of the PL data versus maltose concentration for four different arrangements using 510 nm and 540 nm QDs with MBP-Cy3 using one and two-photon excitation modes. The titration curves demonstrate the equivalence of the sensing arrangements regardless of the QDs used or excitation mode chosen.

Exemplary embodiments describe a method of optically exciting and detecting changes in a nanoscale sensing system comprised of water-soluble luminescent quantum dots and attached biomolecules (e.g., proteins, peptides, oligonucleotides, etc.) which provide the nanocrystals biological specificity for other specific targets or compatibility with biological environments. Multi-photon excitation is used for a variety of purposes: 1) to efficiently and preferentially excite the luminescent QDs in a sample containing other fluorophores (e.g., organic dyes); 2) to improve the penetration depth of the excitation source for in vivo imaging applications; 3) to reduce the illuminated sample volume which allows excellent optical sectioning of a tissue sample; and 4) to minimize potential photo-induced tissue damage caused by long exposure to high intensity sources (e.g., laser irradiation in the visible spectrum). FRET is used as a spectroscopic method of observing molecular-scale changes occurring near the nanocrystal surface. In a molecular sensing arrangement, a shift in the physical position or a change in the local optical properties of a secondary fluorophore (commonly an organic dye molecule) will induce changes in the quantum dot luminescence properties (and often its own as well) by virtue of coupled dipole-dipole interactions between overlapping energy states in the two fluorophores. The combination of multi-photon excitation with FRET provides an attractive platform for efficient sensor designs based on optical imaging, especially in tissue samples.

Exemplary embodiments show a unique advantage of two-photon excitation to probe energy transfer between QDs and conjugated dye acceptors, while investigating the stability of self-assembled QD-peptide conjugates within cells using fluorescence microscopy. Long-term stability of QD-protein conjugates is a crucial requirement in the design of QD-based intracellular sensors. 510 nm emitting QDs were conjugated with polyarginine-containing cell-penetrating peptides (CPP) bearing an N-terminal polyhistidine tract, and subsequently incubated with human embryonic kidney (HEK 293T/17) cells (at a ratio of ˜60 peptides per QD, 50 nM QD) for 1 hour and fixed. These peptides are known to mediate endocytosis of conjugated cargos such as proteins or nanoparticles in a wide variety of cell lines.

In exemplary embodiments, two-photon excited fluorescence images of these cells revealed punctate QD staining consistent with endosomal uptake (FIG. 5A), which was notably absent when the cells were exposed to QDs alone.

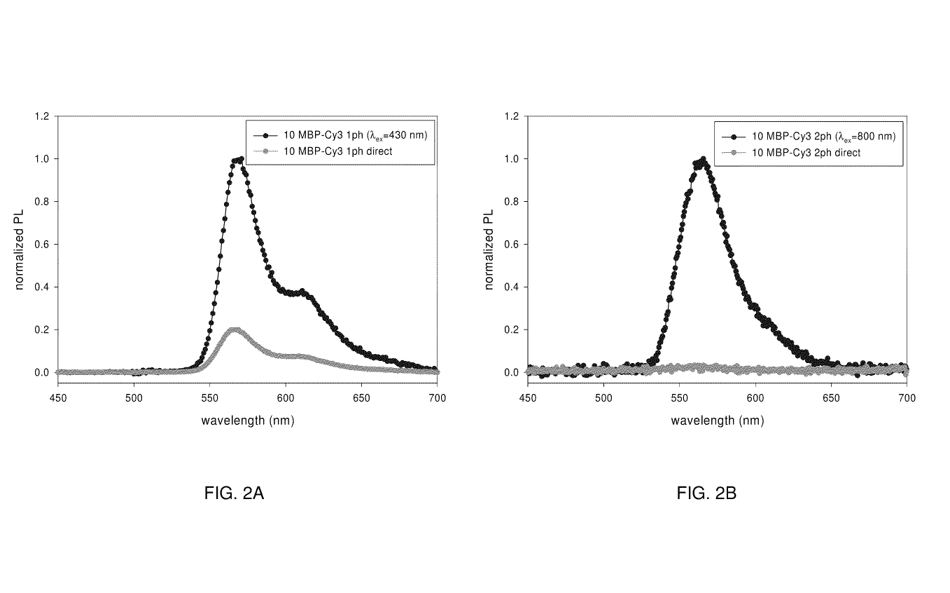

When QDs were conjugated to both CPP and Cy3-labeled peptides (CPP-QD-peptide-Cy3, with 2 Cy3 per QD), efficient FRET was observed from QDs to Cy3, as illustrated in FIG. 2B (top panels).

In contrast, staining of the cells with a mixture of Cy3-labeled transferrin (Tf-Cy3), a commonly used endosomal marker, and unlabeled CPP-QD does not show any evidence of Cy3 fluorescence (FIG. 2A). FIG. 2A illustrates emission spectra from Cy3 using one-photon excitation. In both plots, the bottom curve shows direct excitation of the dye (shown for comparison) and the top curve shows the overall emission signal upon efficient FRET. 540 nm emitting QDs were used in these experiments.

In the latter case, the dye was not directly conjugated to the QDs, thus preventing FRET interactions. In exemplary embodiments the two-photon fluorescence images are compared with the corresponding one-photon epi-fluorescence images using 488-nm excitation (FIG. 2B). While similar images were observed when cells were exposed to CPP-QD and CPP-QD-peptide-Cy3 conjugates, the cells showed bright Cy3 labeling upon exposure to a mixture of CPP-QD and Tf-Cy3. In both plots, in FIG. 2B, the bottom curve is direct excitation of the dye (shown for comparison) and the top curve is the overall emission signal upon efficient FRET. 540 nm emitting QDs were used in these experiments. This demonstrates that significant Cy3 direct excitation occurred upon one-photon excitation at 488 nm (see FIG. 2B).

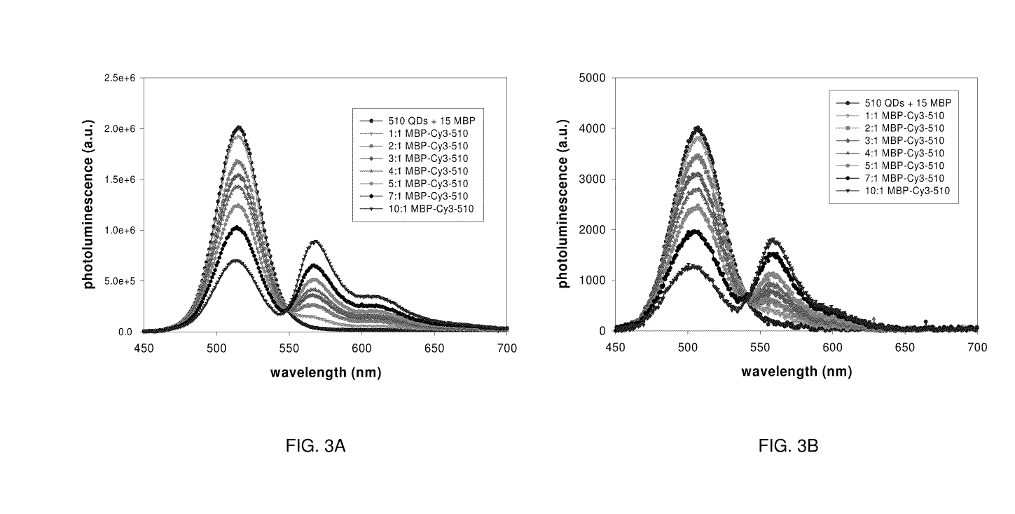

FIG. 3A illustrates a titration sequence showing one-photon emission spectra from a QD-MBP-Cy3 FRET system. Spectra are shown as a function of added dye-labeled protein positioned at the QD surface. PL spectra are shown in arbitrary units.

FIG. 3B illustrates a titration sequence showing two-photon emission spectra from a QD-MBP-Cy3 FRET system. Spectra are shown as a function of added dye-labeled protein positioned at the QD surface. PL spectra are shown in arbitrary units.

As a consequence, it is difficult to distinguish between Cy3 emission due to FRET and direct excitation in a one-photon mode, without additional background correction and image processing. Conversely, PL signal due to direct excitation of the acceptor dye was not observed in a two-photon excitation mode even at 20-fold molar excess relative to the QD donor. The absence of acceptor direct excitation contribution is a unique advantage of two-photon excited FRET using QD donors. While one-photon excitation was only able to probe the presence of QDs and Cy3, two-photon excitation unambiguously reveals co-localized fluorophores and efficient energy transfer from QDs to dyes when QD-peptide-dye conjugates are formed prior to endocytosis. Drawn from this, exemplary embodiments show that the labeled peptides remained stably conjugated to QDs within the endosomal compartments after 72 hours.

Referring to FIG. 7A, FIG. 7B and FIG. 8, at an operation detecting changes 712 (hereafter “the operation 712”) the plurality of measuring instrumentation can be used to detect changes in the nanoscale sensing system 860.

In exemplary embodiments having a two photon excited FRET in a CdSe—Zn QD bioconjugate system, in order for an excitation to be formed, the two photons must arrive at the QD nearly simultaneously (within ˜1 attosecond).

In exemplary embodiments, fluorescence emission from the donor-acceptor system is measured using a suitable photodetection system operating in either a steady-state or time-resolved mode. Steady-state detection essentially integrates the collected fluorescence intensity following a laser pulse or continuous illumination and requires a spectrometer to generate a spectrally-resolved emission spectrum for later analysis. Energy transfer efficiency can be deduced from the change in donor and acceptor signals as compared with proper isolated control samples. In a time-resolved mode, changes in the fluorescence lifetimes of the donor and acceptor pair provide information about the energy transfer efficiency. The measured signals can be spectrally isolated (using a spectrometer or filters), but this is not strictly required for systems that have disparate donor and acceptor lifetimes.

Photoluminescence was detected using an avalanche photodiode, and lifetimes were determined using a time correlated single photon counting (TCSPC) system equipped with a TIMEHARP 200 card and software.

Further, in the first exemplary embodiment, in regard to cell imaging, two-photon cell imaging is performed with a BIO-RAD MRC-1024 MP confocal system using ˜10 mW of 840 nm pulsed excitation (˜80 fs, 80 MHz) at the focal plane of a 60×, 0.9 NA water immersion objective microscope. DAPI, 510 nm QD, and Cy3 signals were separated using 490 nm and 550 nm dichroics. DAPI fluorescence cross-talk was subtracted from the QD channel. One-photon cell imaging of the same samples was performed with an inverted microscope and a PENTAMAX CCD camera. DAPI fluorescence was imaged using 350 nm excitation from a Xe lamp, while QDs and Cy3 were excited with a 488 nm Ar ion laser and spectrally separated using a 565 nm dichroic mirror.

In a second exemplary embodiment, the unique features of two-photon excitation and FRET are combined to implement a reagentless solution-phase sensing assembly specific for the nutrient sugar maltose. In this arrangement, a mutated form of MBP-His was labeled at the unique D41C residue such that binding to maltose induces a conformational transition (to a closed structure), which changes the local environment of Cy3 dye and alters its fluorescence emission. The FRET efficiency is not expected to change as the maltose concentration increases, but rather the PL of the acceptor will vary monotonically due to changes in Cy3 quantum yield. FIG. 6A shows the progression of steady-state fluorescence spectra with increasing maltose concentration. To account for slight variations in excitation efficiency between samples (due to fluctuations in laser power or other factors), from computer processor calculations, we plotted using a computer processor and plotting system, the ratio between the Cy3 peak emission and that of the QD. FIG. 6B shows these ratios as a function of maltose concentration using 510 and 540 nm emitting QDs in one and two-photon excitation modes. As the maltose concentration approaches the equilibrium dissociation constant, K_{D} the Cy3 PL drops dramatically. A K_{D} of 0.8 mM was determined for this sensing assembly demonstrating that the response of the sensor is consistent irrespective of the QD population or the excitation method used. In a two-photon excited system, the QD functions primarily as a light-harvesting fluorophore where exciton energy is transferred from QD to dye. As another species, an alternate maltose sensing scheme was also evaluated using a two-photon excitation mode.

Referring to FIG. 7A, FIG. 7B and FIG. 8, at an operation sensing physiological conditions at the cellular level 714 (hereafter “the operation 714”), exemplary embodiments show that multi-photon excitation of QDs presents several distinct advantages in fluorescence imaging which also extend to FRET-based applications using organic dyes as acceptors. Because of the very large two-photon action cross-sections of luminescent CdSe—ZnS QDs as compared to dyes, direct excitation effects and spectral cross-talk can be reduced to background levels. As a result, detection of molecular-scale changes via FRET can be greatly simplified and improved using two-photon excitation. As nanosensing applications increasingly transition to intracellular environments, multi-photon excitation will allow more accurate targeting of structures and pathways. The interchangeable use of one and two-photon excitation processes in FRET-based sensing allows for the design and characterization of these tools prior to more complicated in vivo implementation. This generalized approach can be used to develop a wide range of unique sensing schemes for real-time intracellular detection, which are compatible with the benefits of multi-photon imaging techniques.

Referring to FIG. 7A, FIG. 7B and FIG. 8, at an operation return/end 715, operations of the method 700 can iteratively be repeated or selectively be repeated at any given operation of the method 700, until the operations of the method 700 are completed or the operations can continue until a signal from the user is initiated and/or received causing the system 800 and the method 700 to stop and/or end.

Referring to FIG. 8, in accordance with exemplary embodiments, the system 800 embodies and implements the various methods, procedures and operations of the method 700 in the structure of computer executable program code, computer executable and computer readable media and other hardware, firmware and software modules, network applications and interface platforms, upon which the method 700 is carried out within the technological arts.

Referring to FIG. 8, in accordance with exemplary embodiments, the data control bus 898 communicatively connects the computer processor 829 to plurality of measuring instrumentation, where the data control bus 898 can be a wide area communications network, including an Internet or an extranet or the network 829 can be a local area network, including an intranet. The plurality of measuring instrumentation can include host computers, storage devices, such as tape drives, disc drives operating individually or in storage library farms.

In exemplary embodiments, the system 800 and the method 700 illustrated in FIG. 8, FIG. 7A and FIG. 7B respectively can be partially and/or fully implemented in software, firmware or hardware or a combination of each. According to exemplary embodiments, the method 700 can be partially and/or fully implemented in software, as executable program code, which comprises an ordered listing of executable instructions for implementing logical functions, and which is executed by either special or general purpose digital computers including a PDA, a personal computer, a workstation, a minicomputer or a mainframe computer, a controller or some other measuring instrumentation.

In exemplary embodiments, the system 700 implements a general purpose digital computer designated as the computer processor 829. The computer processor 829 is a hardware device for executing software implementing the method 700. The computer processor 829 can be any custom made or commercially available, off-the-shelf processor, a central processing unit (CPU), one or more auxiliary processors, parallel processors, graphics processors (or such as graphics processors operating as parallel processors), a semiconductor based microprocessor, in the form of a microchip or chip set, a macroprocesssor or generally any device for executing software instructions. The system 800 when implemented in hardware can include discrete logic circuits having logic gates for implementing logic functions upon data signals, or the system 800 can include an application specific integrated circuit (ASIC).

In exemplary embodiments, referring to FIG. 8, the memory 808 and the dynamic repository 809 and storage devices can include any one of or a combination of volatile memory elements, including random access memory (i.e., including RAM, DRAM, SRAM and/or SDRAM) and non-volatile memory elements including read only memory (i.e., ROM, erasable programmable read only memory, electronically erasable programmable read only memory EEPROM, programmable read only memory PROM, and/or compact disc read only memory CDROM or FLASH memory or cache) magnetic tape, disk, diskette, cartridge, cassette and/or optical memory. The memory 808 can have an architecture where various components are situated remotely from one another, but can be accessed by the computer processor 829, either directly and/or locally and/or remotely through/over the data control bus 898 or various communications networks.

While the exemplary embodiments have been particularly shown and described with reference to preferred embodiments thereof, it will be understood by those skilled in the art that the preferred embodiments including the first exemplary embodiment, the second exemplary embodiment and the third exemplary embodiment have been presented by way of example only, and not limitation; furthermore, various changes in form and details can be made therein without departing from the spirit and scope of the invention. Thus, the breadth and scope of the present exemplary embodiments should not be limited by any of the above described preferred exemplary embodiments, but should be defined only in accordance with the following claims and their equivalents. All references cited herein, including issued U.S. patents, or any other references, are each entirely incorporated by reference herein, including all data, tables, figures, and text presented in the cited references. Also, it is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

What is claimed is:

1. A method of sensing physiological conditions in biological applications using a multi-photon excitation system having a laser source and automated measuring instrumentation in a nanoscale sensing system, the method comprising:
preparing a plurality of acceptors in the nanoscale sensing system;
directly optically exciting, with the laser source, a plurality of donors and the plurality of acceptors in the nanoscale sensing system, wherein the laser source includes one of pulsed and continuous wave excitation sources;
transferring energy, in an energy transfer process, between the plurality of donors and the plurality of acceptors;
investigating, with automated measuring instrumentation, stability of self-assembly of the plurality of acceptors, within the plurality of donors;
detecting, using a photo detector, changes in the nanoscale sensing system, wherein detecting provides one of a biological specificity for specific targets and compatability with biological environments; and
sensing, using a spectrometer, fluorescence spectra of physiological conditions at a cellular level, wherein sensing, using the spectrometer, fluorescence spectra of physiological conditions includes a transducing of signals into immunoassays, clinical diagnostics and cellular imaging to provide treatment to biological subjects at the cellular level, wherein the transducing of signals by the multi-photon excitation system removes corrupting influences of directly optically exciting the plurality of acceptors, while preferentially directly optically exciting the plurality of donors.

2. The method of claim 1, wherein the plurality of acceptors are fluorophores including one of a plurality of organic dyes, and a plurality of non biological molecules including one of a plurality of metal complex and a plurality of polymers.

3. The method of claim 1, wherein the energy transfer process between the plurality of donors and the plurality of acceptors includes performing one or more of a fluorescence resonance energy transfer (FRET) process and performing a Dexter energy transfer process and performing a surface energy transfer (SET) process.

4. The method of claim 1, wherein treatment to biological subjects at the cellular level, includes treatment in biological tissue samples of plant, animal, and human patients.

5. The method of claim 1, wherein the plurality of donors include a plurality of quantum dots bound with a plurality of biological molecules.

6. The method of claim 5, wherein use of the plurality of quantum dots minimizes photo oxidation of donors bound with the plurality biological molecules.

7. The method of claim 6, wherein the plurality of biological molecules include one of proteins, oligonucleotides, and peptides.

8. A system for sensing physiological conditions in biological applications in a nanoscale sensing system, the system comprising:
a nanoscale sensing system associated with a plurality of automated measurement instrumentation for preparing a plurality of acceptors in the nanoscale sensing system, wherein one automated measurement instrument of the plurality of automated measurement instrumentation is a photon counting system having a computer processor;
a pulsed laser source communicatively coupled to the nanoscale sensing system and the plurality of automated measurement instrumentation, wherein the nanoscale sensing system includes a nanocrystal structure;
an input/output device;
a data control bus, wherein the data control bus communicatively couples the computer processor of the photon counting system to the pulsed laser source, to the nanoscale sensing system, and to the plurality of automated measurement instrumentation, and to the input/output device; and
a memory, residing in the computer processor, having a dynamic repository and a program unit containing a computer readable and a computer executable program; wherein when the computer executable program is executed by the computer processor, the computer executable program causes the system for sensing physiological conditions in biological applications to perform operations including:
directly optically exciting, with the pulsed laser source, a plurality of donors and the plurality of acceptors in the nanoscale sensing system, wherein the pulsed laser source includes one of pulsed and continuous wave excitation sources;
transferring energy, in an energy transfer process, between the plurality of donors and the plurality of acceptors;
investigating, with automated measuring instrumentation, stability of self-assembly of the plurality of acceptors, within the plurality of donors;
detecting, using a photo detector, changes in the nanoscale sensing system, wherein detecting provides one of a biological specificity for specific targets and compatability with biological environments; and
sensing, using a spectrometer, fluorescence spectra of physiological conditions at a cellular level, wherein sensing, using the spectrometer, fluorescence spectra of physiological conditions includes a transducing of signals into immunoassays, clinical diagnostics and cellular imaging to provide treatment to biological subjects at the cellular level, wherein the transducing of signals by the multi-photon excitation system removes corrupting influences of directly optically exciting the plurality of acceptors, while preferentially directly optically exciting the plurality of donors.

9. The system of claim 8, wherein the plurality of acceptors are fluorophores including one of a plurality of organic dyes, and a plurality of non-biological molecules including one of a plurality of metal complex and a plurality of polymers.

10. The system of claim 8, wherein the energy transfer process between the plurality of donors and the plurality of acceptors includes one or more of a fluorescence resonance energy transfer (FRET) process and performing a Dexter energy transfer process and performing a surface energy transfer (SET) process.

11. The system of claim 8, wherein treatment to biological subjects at the cellular level, includes treatment in biological tissue samples of to plant, animal, and human patients.

12. The system of claim 8, wherein the plurality of donors include a plurality of quantum dots bound with a plurality of biological molecules.

13. The system of claim 12, wherein application of the plurality of quantum dots minimizes photo oxidation of donors bound with the plurality biological molecules.

14. The system of claim 13, wherein the plurality of biological molecules include one of proteins, oligonucleotides and peptides.

15. A method of sensing physiological conditions in biological applications using a two photon excitation system having a continuous wave laser source and automated measuring instrumentation in a nanoscale sensing system, the method comprising:
preparing a plurality of donors and acceptors in the nanoscale sensing system;
optically exciting, with the continuous wave laser source, the plurality of donors and acceptors;
transferring energy, between the plurality of donors and acceptors;
investigating, with automated measuring instrumentation, stability of self assembly of acceptors bound to donors;
detecting changes in the nanoscale sensing system; and
sensing, using a spectrometer, fluorescence spectra of physiological conditions at a cellular level, wherein sensing, includes a transducing signals into immunoassays, clinical diagnostics and cellular imaging, while removing corrupting influences of optically exciting acceptors, and while preferentially optically exciting donors.

16. The method of claim 15, wherein transducing signals into immunoassays, clinical diagnostics, and cellular imaging includes sensing physiological conditions in biological tissue samples for one of fixed and living biological tissue samples and providing treatment to biological tissue samples for one of fixed and living biological tissue samples.

17. The method of claim 15, wherein preparing a plurality of donors and acceptors includes staining a plurality of biomolecules with dye in the nanoscale sensing system.

18. The method of claim 15, wherein investigating, with automated measuring instrumentation, stability of self assembly of acceptors bound to donors includes investigating stability of self assembly of quantum dot peptide conjugates, within the plurality of biomolecules and the two photon excitation system using FRET causing a reagentless solution phase sensing assembly specific for nutrient sugar maltose, which changes a local environment of Cy3 dye and alters fluorescence emission of a photoluminescence contribution.

19. The method of claim 18, wherein nutrient sugar maltose includes engineered variants of E. coli maltose binding proteins appended with a C-terminal polyhistidine tract to cause metal affinity, driven self assembly of DHLA functionalized quantum dots.

20. The method of claim 19, wherein an overall MBP-to-QD ratio is maintained at a 15:1 ratio.
